(19) United States
(12) Patent Application Publication
(10) Pub. No.: US 2016/0045326 A1
(43) Pub. Date: Feb. 18, 2016

(54) INTERBODY SPACER SYSTEM

(71) Applicants: Eric Hansen, Arlington, TX (US); Scott Hay, Arlington, TX (US)

(72) Inventors: Eric Hansen, Arlington, TX (US); Scott Hay, Arlington, TX (US)

(21) Appl. No.: 14/461,448

(22) Filed: Aug. 18, 2014

(51) Int. Cl. A61F 2/44; A61F 2/46

(52) U.S. Cl. CPC A61F 2/447; A61F 2/4611; A61F 2002/4475; A61F 2002/30518

(57) ABSTRACT

A device, system, and method are disclosed that supplements a human spinal column's structure by replacing a damaged intervertebral disc with a machined spacer comprising a movable pair of blades that rotate to expand from the spacer in-situ to interlock with a patient's vertebras, and a locking system that provides secondary protection from blade movement within the spacer.

### TECHNICAL FIELD OF THE INVENTION

The present disclosure relates generally to interbody spacers used to repair damaged intervertebral discs.

### BACKGROUND OF THE INVENTION

Human spines comprise movable vertebrae, with cushioning disks between them. These intervertebral disks can be damaged in many ways, and they degenerate with age. Damage to these disks can often be debilitating and cause excruciating pain.

The medical community has worked for decades to develop methods of repairing damaged spines and alleviate the associated pain. Ruptured intervertebral disks are a very common back problem, caused when a disk is ruptured or loses the fluid within it, and thus can no longer provide a proper cushion between vertebras.

One common method of addressing this damage is to employ spinal fusion, a surgical technique used to facilitate the growth of bone between two vertebrae. The procedure involves implanting an “interbody”, packed with grafting material into the disc space, to stabilize the spine while bone grows in between two vertebrae. As the bone graft material heals, one long bone is formed with the adjacent vertebrae.

The interbody, a spacer most often made from titanium or polyetheretherketone (“PEEK”) material, is set between the vertebras on either side of the damaged disk.

Recent developments have resulted in the “stand alone” interbody spacer, as discussed in U.S. Pat. 8,328,870 (Patel, et al.), in which the spacer contains its own support means of fixation. Previous to this industry development, doctors had to add screws and other devices to the spacer to keep it in place, relative to the vertebra.

The industry has not fully adopted the stand alone spacer, as current devices still struggle to stay in place. One example of a modern device is shown in U.S. Pat. No. 8,273,127, where the spacer has a load-bearing piece, and a second piece designed to prevent the spacer from migrating, as well as two screws that extend to engage in the vertebra both above and below the spacer.

An additional need by the medical industry is a device that can be implanted with minimal invasion to a patient's body.

The industry still seeks a stand-alone interbody spacer that can reliably be installed using a minimally invasive procedure, such that a patient's spine can fuse around the spacer without continuous medical attention to prevent spacer movement.

### SUMMARY OF THE INVENTION

The present disclosure provides a device, system, and method to supplement a human spinal column's structure by replacing a damaged intervertebral disc with a PEEK spacer comprising a movable pair of blades. The blades sit within the envelope of the spacer body during the implantation surgery until the spacer is properly set. After properly situated, the medical team actuates the rotation of the blades, which then extend both up and down from the spacer's body, providing an interlock with the vertebras above and below the spacer. The blades are locked in place, preventing the spacer from migrating out of position.

Novel and inobvious aspects of the invention comprise a new locking feature for the interlocking blade and blade shape. Other features and advantages of the present disclosure will be apparent to those of ordinary skill in the art upon reference to the following detailed description taken in conjunction with the accompanying drawings.

### BRIEF DESCRIPTION OF THE DRAWINGS

For a better understanding of the disclosure, and to show by way of example how the same may be carried into effect, reference is now made to the detailed description along with the accompanying figures in which corresponding numerals in the different figures refer to corresponding parts and in which:

FIG. 1 depicts an orthogonal view of one embodiment of an 18 mm Spacer Body 100.

FIG. 2 depicts a front view of the embodiment of an 18 mm Spacer Body 100 (without hidden lines).

FIG. 3 depicts a left view of the embodiment of an 18 mm Spacer Body 100 (without hidden lines).

FIG. 4 depicts the sectional view of an 18 mm Spacer Body 100 along section lines A-A of FIG. 2.

FIG. 5 depicts a top view of an 18 mm embodiment as shown in FIG. 1-4.

FIG. 6 depicts a section view defined by FIG. 5 along section lines H-H.

FIG. 7 depicts a bottom view of the embodiment of a 10 mm embodiment of the Interbody Spacer Assembly 10 with the Blade Pair 300 in the open position.

FIG. 8A shows an orthogonal view of a Center Shaft 200 for the embodiment depicted in FIG. 1.

FIG. 8B shows the torx head of the Center Shaft 200 for the embodiment depicted in FIG. 1.

FIG. 8C shows a side view of the Center Shaft 200 for the embodiment depicted in FIG. 1.

FIG. 8D shows a sectional view of the Center Shaft 200 for the embodiment depicted in FIG. 1 along the shaft's central axis.

FIG. 9A shows an orthogonal view of a Washer Nut 500 for the embodiment depicted in FIG. 1.

FIG. 9B shows a top view of a Washer Nut 500 for the embodiment depicted in FIG. 1.

FIG. 9C shows a side view of a Washer Nut 500 for the embodiment depicted in FIG. 1.

FIG. 9D shows a sectional view of a Washer Nut 500 along section lines A-A of FIG. 9B.

FIG. 9E shows a bottom view of one embodiment of a Washer Nut 500.

FIG. 10A shows an orthogonal view of the embodiment of a Lock Sleeve 400.

FIG. 10B shows a rear view of the embodiment of a Lock Sleeve 400 shown in FIG. 10A.

FIG. 10C shows a front view of the embodiment of a Lock Sleeve 400 shown in FIG. 10A.

FIG. 10D shows a sectional view defined by the section lines A-A of FIG. 10B.

FIG. 10E shows a sectional view defined by the section lines B-B of FIG. 10C.

FIG. 10F shows a sectional view defined by the section lines C-C of FIG. 10C.

FIG. 11A shows an exploded view of an 18 mm embodiment of an Interbody Spacer 10.

FIG. 11B shows an orthogonal view of the embodiment of a Blade Pair 300 as shown in FIG. 11A.

FIG. 11C shows a front view of the embodiment of a Blade Pair 300 as shown in FIG. 11A.

FIG. 11D shows a rear view of the embodiment of a Blade Pair 300 shown in FIG. 11A.

FIG. 11E shows a right side view of the embodiment of a Blade Pair 300 shown in FIG. 11A.

FIG. 11F shows a cross-sectional right side view of the embodiment of a Blade Pair 300 defined by the section lines A-A of FIG. 11C.

FIG. 11E shows a sectional view defined by the section lines B-B of FIG. 10C.

FIG. 11G shows the detail sectional area defined by the circular area B of FIG. 11F.

FIG. 11H shows a top view of the embodiment of a Blade Pair 300 shown in FIG. 11A.

FIG. 11I shows a sectional view defined by the section lines G-G of FIG. 11H.

FIG. 12 shows a rear view of an embodiment of an Interbody Spacer 10 as embodied in the previous figures.

FIG. 13 shows the construction details of a Spike 210.

FIG. 14 shows a side view of an Interbody Spacer Assembly 10 with Blade Pair extended from the Spacer Body 100.

It should be noted that these drawings show two different embodiments, a 10 mm and an 18 mm version.

These two embodiments are mere exemplars, and not intended to represent the extent of the invention.

### DETAILED DESCRIPTION OF THE INVENTION

While the making and using of various embodiments of the present disclosure are discussed in detail below, it should be appreciated that the present disclosure provides many applicable inventive concepts, which can be embodied in a wide variety of specific contexts. The disclosure is primarily described and illustrated hereinafter in conjunction with various embodiments of the presently-described systems and methods. The specific embodiments discussed herein are, however, merely illustrative of specific ways to make and use the disclosure and do not limit the scope of the disclosure.

The Shark Fin PEEK Spacer Method is intended for spinal fusion procedures in skeletally mature patients with degenerative disc disease (ODD) at one or two contiguous levels in the lumbar spine (L2-S1). DOD is defined as back pain of discogenic origin with degeneration of the disc confirmed by patient history and radiographic studies—DOD patients may also have a spondylolisthesis at the involved levels and may also have had a previous non-fusion surgical history.

The Shark Fin PEEK Spacer Method is intended to address this type pathology and is designed to host autograft when implanted. It is not uncommon for patients to have undergone a regimen of at least six months of non-operative treatment prior to being recommended for the Shark Fin Spacer Method.

The Shark Fin PEEK Spacer Method offers a surgeon a reliable, integrated standalone AUF (Anterior Lumbar Inter-body Fusion) solution that is simple to implant versus other similar type implants. The Shark Fin PEEK Spacer Method also meets the preferences of surgeons to improve care for diverse patient anatomies. This design, like others, includes a variety of implant heights and widths and lordotic angles to allow the AUF approach to harmonize to a given patient's anatomy. And because the Keel Locking Method eliminates lock-down screws this style implant is especially advantageous for difficult cases in the L5 to S1 region of the spine.

To date, this new standalone cage design has received to positive feedback in its concept form from surgeons experienced in the art of Standalone AUF procedures—it easily meets patient anatomy and may provide one of the more time efficient surgical procedures versus other AUF devices in its class. A variety of materials such as PEEK OPTIMA, titanium, cobalt, chrome, carbon fiber, PEKK, etc. can define the body and related mechanical components respectively while an anterior plate and other supplemental add-on devices such as a buttress plate could be used to reinforce the construct predominantly defined by this device.

The Locking Keel is a novel component that provides the surgeon with a simplified two-step implant locking procedure to secure an Interbody Spacer Assembly 10 into final position to prevent migration.

As seen in the drawings and currently embodied, the Interbody Spacer Assembly 10 comprises a Spacer Body 100, a Center Shaft 200 which an operator turns while it is engaged with a Blade Pair 300 which is held in place by a Lock Sleeve 400 and Washer Nut 500, and Spikes 600 on the Spacer Body 100 to exterior.

The Spikes 600 on the Upper Surface 110 and Lower Surface 120 of the Interbody Spacer Assembly 10 prevent migration of the Assembly 10 after installation.

Upper and Lower Surfaces 110, 120 possess an angle called a lordotic angle to allow the implant to match patient specific segmental angular anatomy with lordotic angles ranging between 0 and 7 degrees.

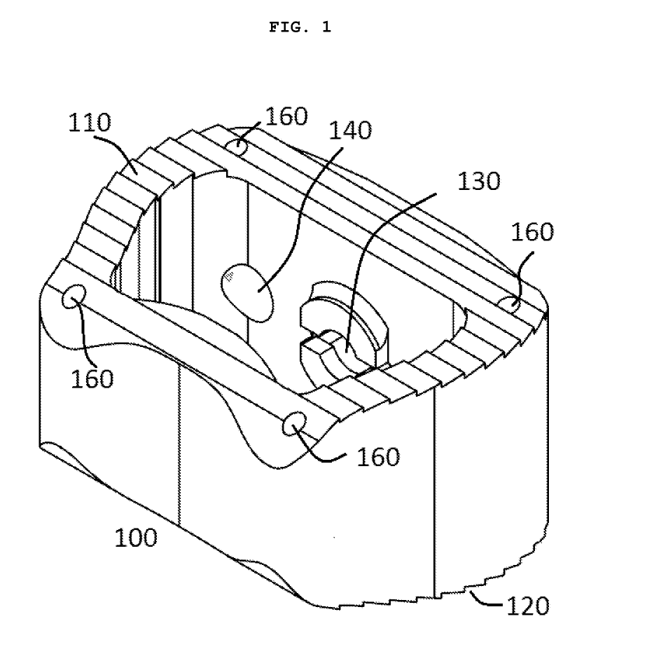

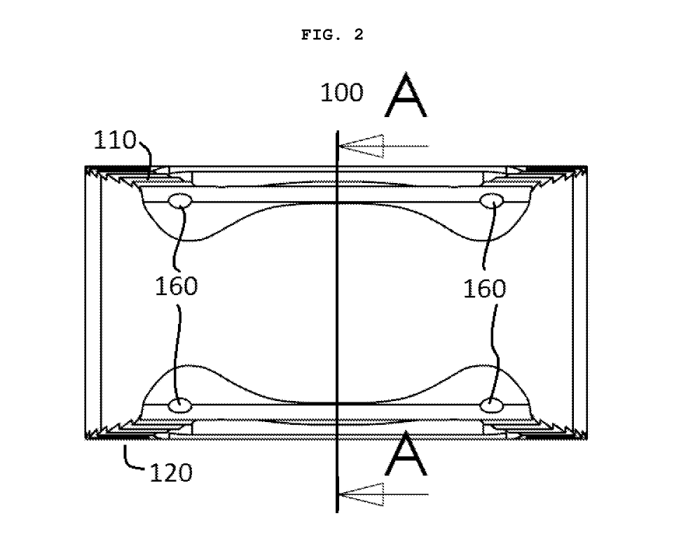

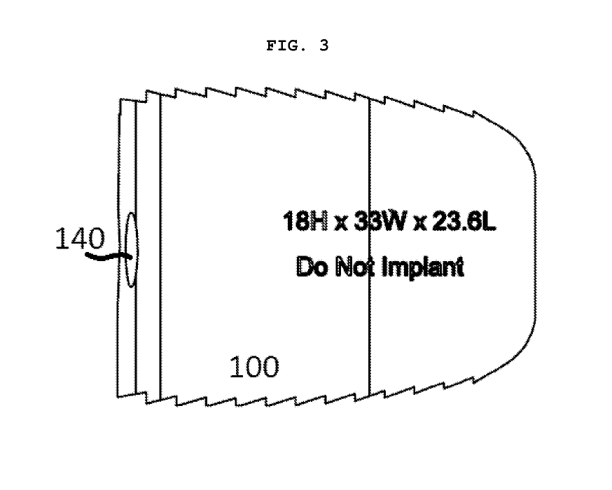

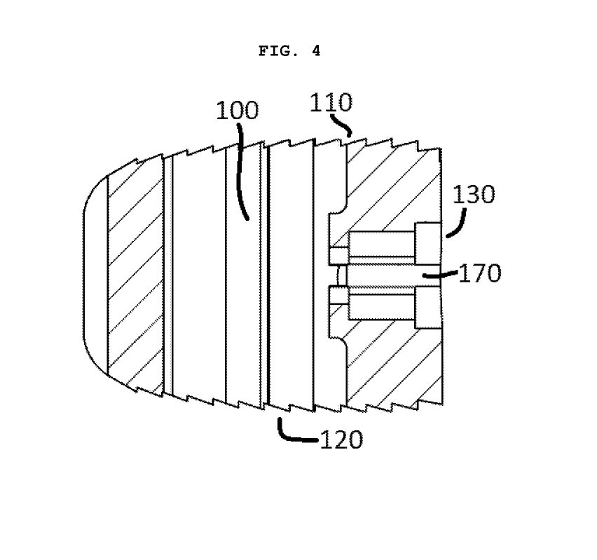

The upper and lower surface Spikes 600 can include an ovoid shape as well (a slightly convex curve), as shown in FIGS. 1, 5, 7 and 11A, to also help match the implant to patient anatomy.

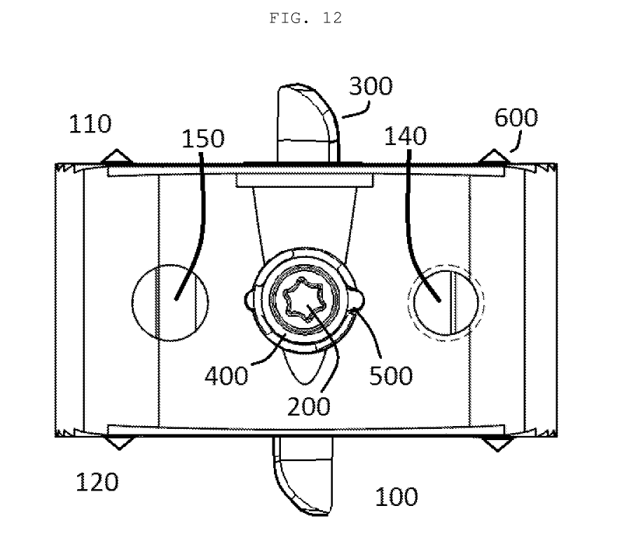

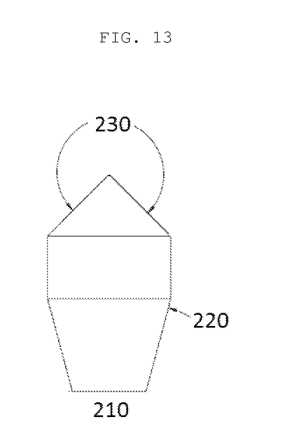

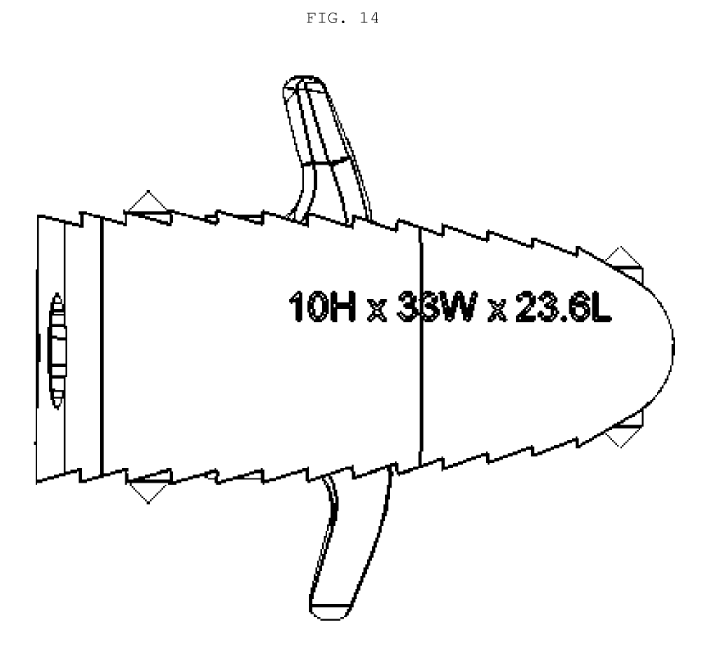

The wide central opening in the Shark Fin PEEK Spacer Body 100 essentially is used to hold optimal graft material. As seen in FIG. 12, after the Blades 300 are turned to their open position extending above and below the Spacer Body 100, a user can inject additional materials into the volume encased by the Body 100 through the Access Hole 150, even mounting instruments on the Body 100 in the threaded Instrumentation Mount 140.

The Shark Fin Spacer Assembly 10 can be constructed in many sizes, most notably in the range of range of 10 mm to 22 mm in height and 32 mm to 36 mm in width, but is not restrained to those sizes.

Tantalum X-ray markers may be located on the upper and lower surfaces of the implant body to provide clear radiographic identification. However, given the Keel-Plate design strategy, fewer implant markers may be necessary, such as using the markers only on the distal part of the implant body.

Two lateral openings are built into the Spacer Body 100. The Instrumentation Mount 140 allows for insertion tool attachment, providing a threaded hole in the Body 100. Due to mechanical implant locking design multiple implant deliver angles can be accommodated. The Access Hole 150 allows medical personnel to have access to the volume encased by the Body 100.

The Keel Locking Blade is a distinct construction employing a slight cord-wise twist definition to increase blade deflection resistance, which mitigates blade failure during service life and helps surgeons to more easily and securely lock the implant into a permanent location.

The Blade Pair 300 is constructed so that when the pair is turned in the open position (such that the blades are extended above and below the installed Interbody Spacer Assembly 10), the unthreaded Access Hole 150 in the rear of the Assembly allows medical personnel to insert biologics into the internal volume enclosed by the Spacer Body 100.

A threaded Instrument Mount Hole 140 is also positioned on the rear of the Spacer Body, as shown on FIG. 12. This threaded hole acts as a mounting position for insertion tool, biologic injection guns, and other similar medical gear.

The chord-wise twist definition of the Blade Pair 300 is defined similar to radially extending a jack screw thread pattern (i.e. if the Blades' chord-wise planar surfaces were projected outward in a radial manner, the resulting geometry would describe a typical jack screw thread pattern; this not only creates maximum blade deflection resistance, but also creates subtle mechanical leverage during blade rotation to better purchase the implant into its final resting position.

This design strategy provides a constant “sweep angle” along the length of each blade on Blade Pair 300 to optimize blade lengthwise stiffness and reduce blade failure likelihood anywhere along the blades' span, including the blades' root.

The leading edge of each blade of the Blade Pair 300 will have a sharpened edge to allow easy penetration into the boney upper and lower plates of the vertebra when the Blade Pair 300 is rotated from its resting “near horizontal” position into a locked “vertical” position. The edge geometry may have serrated edges or smooth edges, or smooth edges with a partial serration so the Blade Pair can more easily penetrate a user's surface with minimal surface fracture.

The root of each of the blades of the Blade Pair 300 will possess a graduating thickness where maximum bending moments occur to prevent the blade from fatigue due to the blades' surrounding cyclical environmental loading conditions.

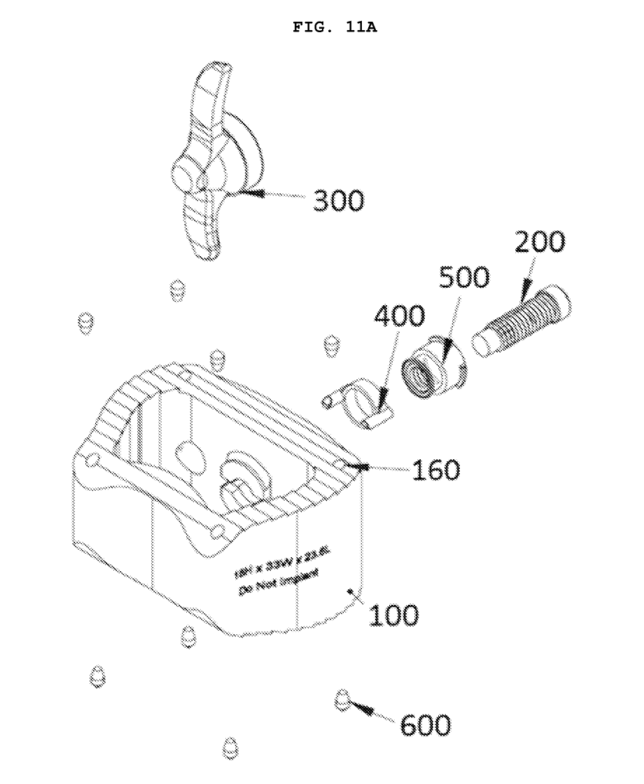

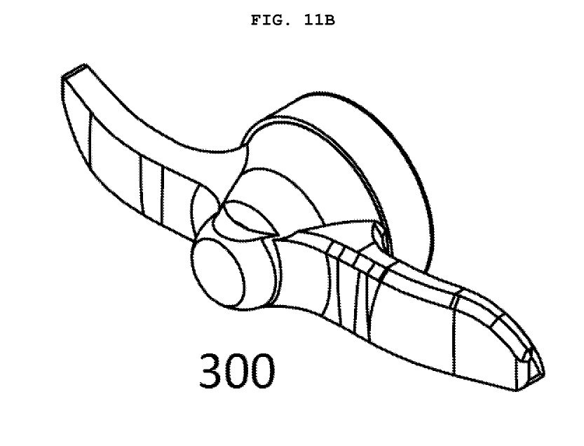

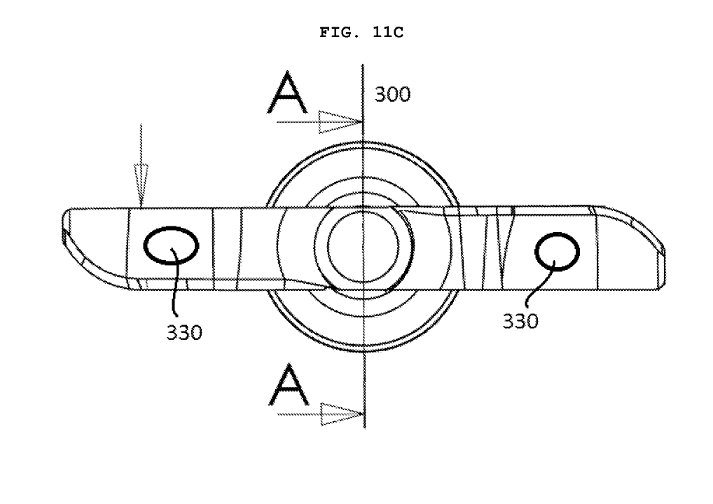

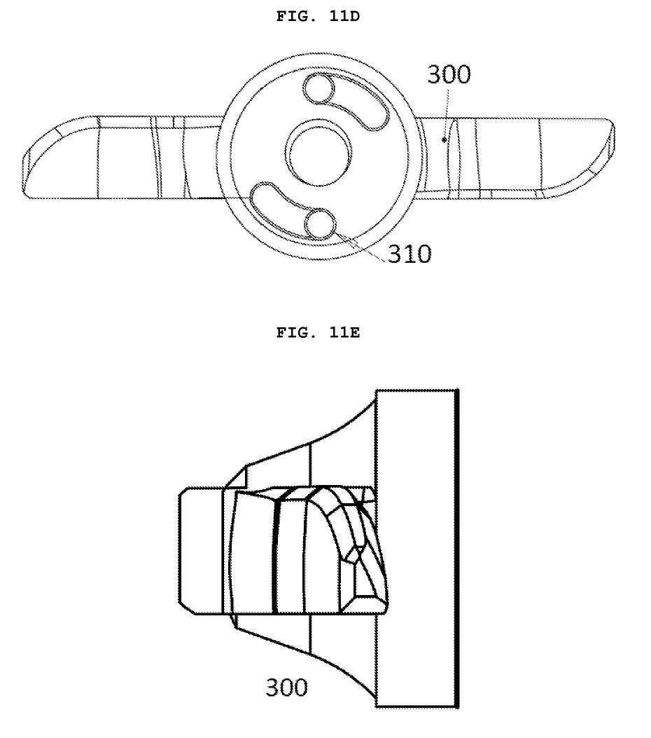

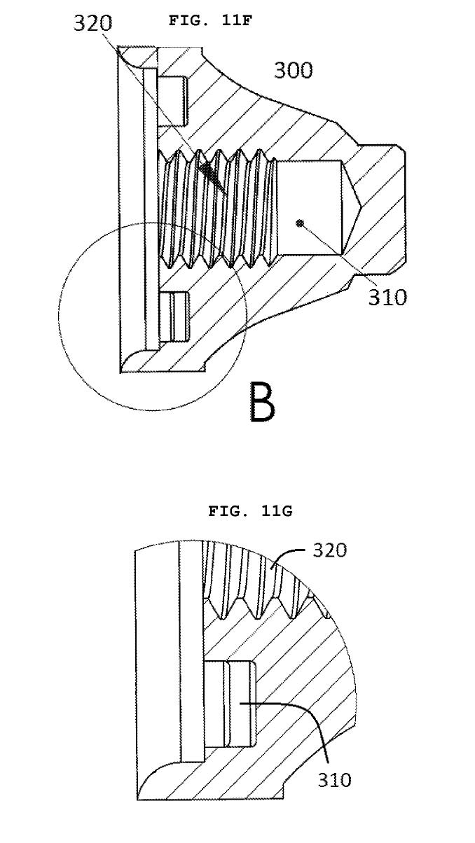

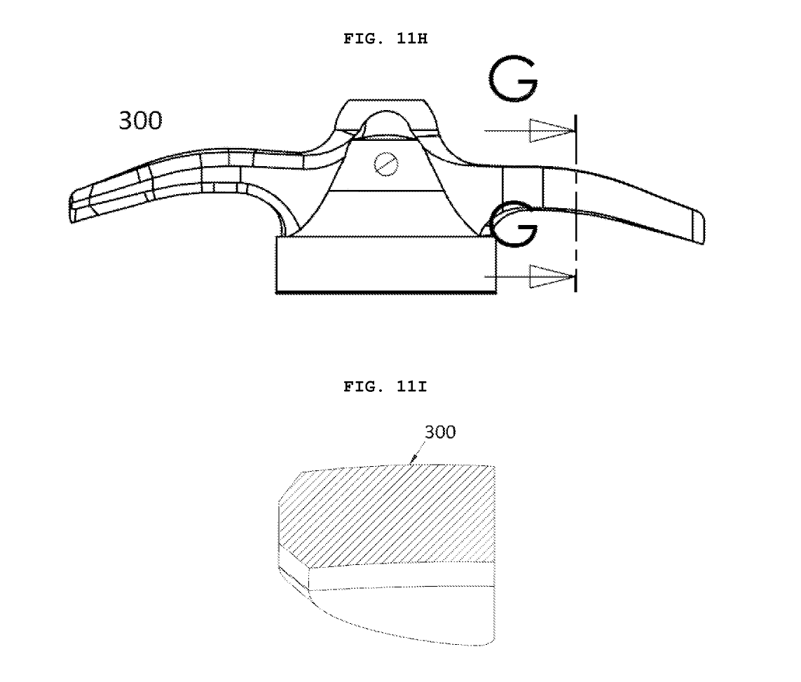

The Blade Pair 300 may possess optional Blade Pair Clearance Holes 330 in its body to provide apertures for bone growth, as shown in FIG. 11C. The size of the aperture is a discretionary design option with its size and location to be ideally located so as not to reduce the structural integrity of the blade design. FIG. 11C shows two differing shapes and locations for Clearance Holes 330, but this is a mere example and is not intended to be limiting.

The hub geometry may or may not have a “keyed” hole and slot to allow the Blade Pair 300 to be rotated by a Center Shaft 200, though the embodiment. In the current design, a press fit-welded pin will supplement and create the rigid mechanical connection between the blade and the shaft.

The lengthwise distance of the Keel or Blade Pair 300 will be variable and will always be a minimum of 10 mm longer than the height of the implant body that it resides within (i.e., 5 mm beyond than the upper and lower profile horizon of the implant body).

The Blade Center Shaft 200 is designed to rotationally articulate the Blade Pair 300 from a resting or closed position to a vertical locked position to prevent the Interbody Spacer 10 from migration after installation.

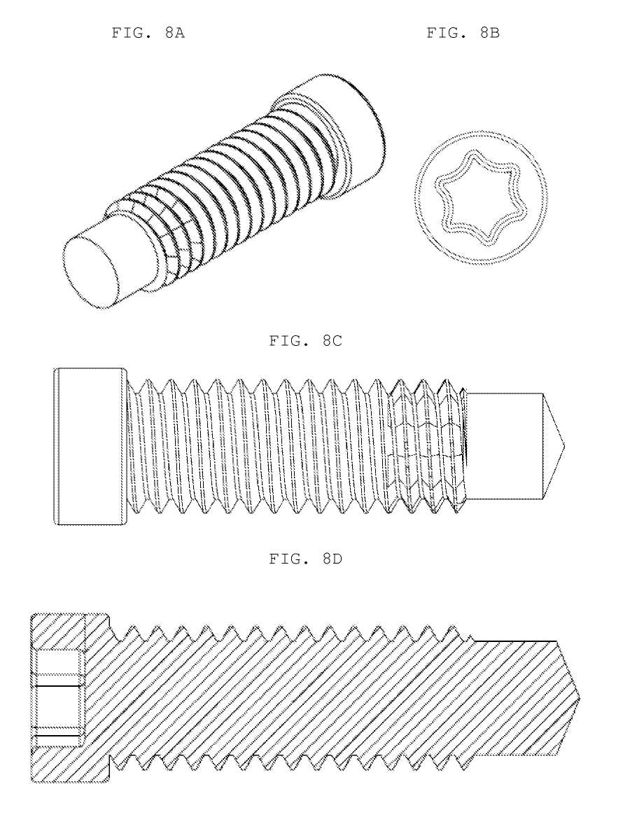

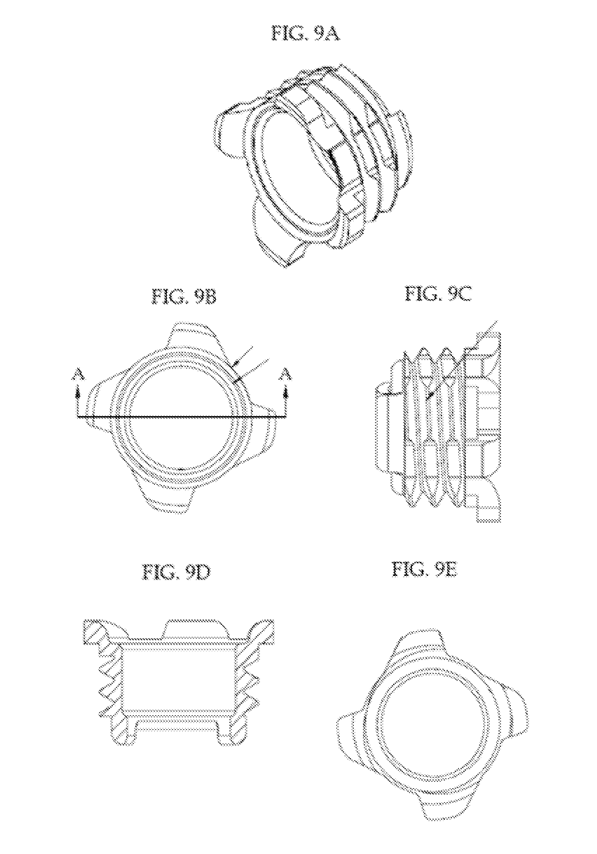

As shown in FIG. 9A-9E, the proximal portion of the Washer Nut 500 possesses a four fingered expansion collar that is designed to expand under the influence of an inner screw where the four fingers expand outward and into the Spacer Body 100 to create an almost cold weld mechanical interference.

The bearing surface of the Center Shaft 200 to the Center Shaft Channel 130 is a high-precision cam geometry that allows for easy rotation within the Spacer Body 100, and provides the predominant interface into the Spacer Body 100 where the majority of mechanical loading is translated.

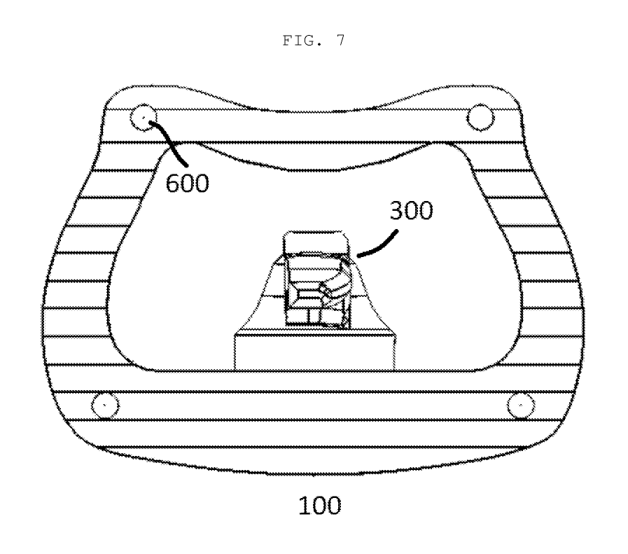

As seen in FIGS. 8A & 8C, just past the bearing surface is a region that may or may not be threaded that assists in the connectivity of the shaft to the blade. In early models, a press fit pin is used for this connectivity which may be optionally welded for final fastening. This assembly strategy precludes possible mating separation and provides a redundant mating method for the blade shaft interface.

The largest lengthwise portion of the shaft that possesses a simple outer diameter is designed as a near “press-fit” feature that allows the shaft to engage the rotational Blade Pair 300 to reinforce the rigid interface required between the Shaft 200 and the Blade Pair 300.

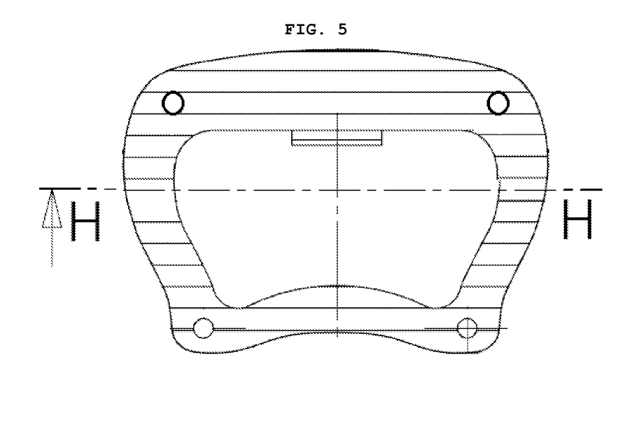

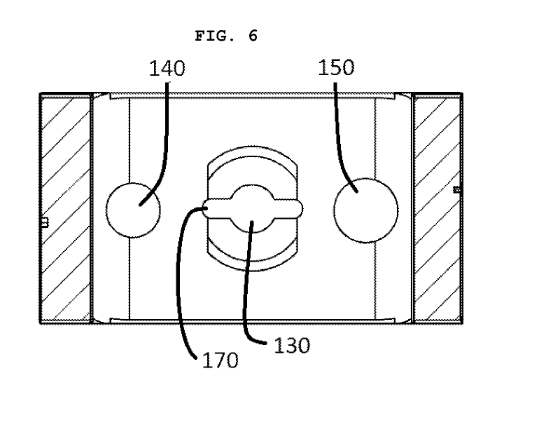

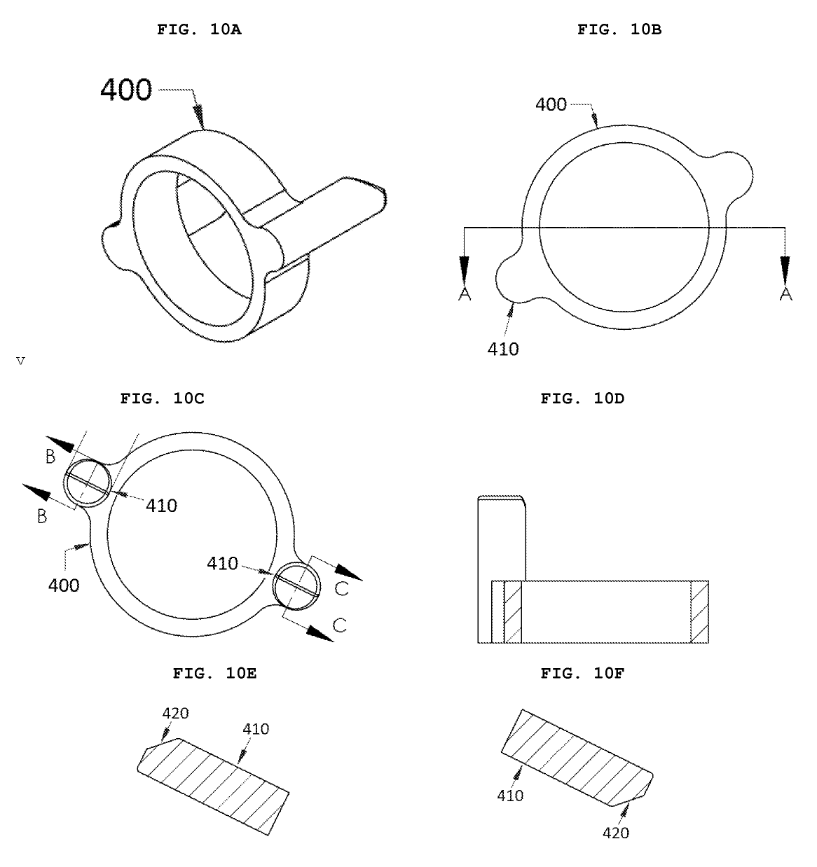

This invention includes an innovative concept of an interlocking Lock Sleeve 400 as shown in FIG. 10A. Two Lock Sleeve Fingers 410 extend from the Lock Sleeve 400. During the installation of the invention, a user turns the Center Shaft 200. The Blades 300 are pressed onto the Center Shaft 200 so they turn as one unit to the open position, extending vertically above and below the Spacer Body 100. As shown in FIG. 11A, the Center Shaft 200 is not cinched tight into the Spacer Body. Instead, movement of the Center Shaft 200 is prevented by a Washer Nut 500 that is tightened on the Center Shaft to the Spacer Body 100. While it is tightened, however, it also pushes the Lock Sleeve Fingers 410 through the Lock Finger Channel 170 constructed in the Spacer Body 100 (as seen in FIG. 6). FIG. 11D shows the Lock Finger Indent, which accepts the Lock Sleeve Fingers 410 to hold them in place. This interlocking finger/indent construction provides secondary protection from Blade 300 movement within the Body 100 should the washer loosen.

The invention is superior to other industry offerings because this construction requires no bone screws and allows for easy load sharing.

All embodiments described herein are presented for purposes of illustration and explanation only. These descriptions of one embodiment are not intended to be limiting to the embodiments described. Those skilled in the relevant art will be able to create other embodiments based on this disclosure and the claims that are attached with this application

The figures of this patent application include the following components and nomenclature:

10 Interbody Spacer Assembly

100 Spacer Body

110 Upper Surface

120 Lower Surface

130 Center Shaft Channel

140 Instrumentation Mount

150 Access Hole

160 Spike Mounting Holes

170 Lock Finger Channel

200 Center Shaft

210 Spike

220 Spike Angle of Installation

230 Spike Angle of Contact

300 Blade Pair

310 Lock Finger Indent

330 Blade Pair Clearance Holes

400 Lock Sleeve

410 Lock Sleeve Finger

420 Lock Sleeve Finger Engagement

500 Washer Nut

600 Spike

The inventors claim:

1. An interbody spacer system, comprising a hollowed spacer body which replaces a damaged intervertebral disk, said spacer body with a pair of movable blades fixed within the spacer body that can be turned and are constructed to extend above and below the spacer body when oriented vertically to interlock with the vertebras directly above and below the spacer body, said blades locked in place once the spacer body is installed.

2. An interbody spacer system as in claim 1, with the addition feature that the spacer cage is constructed with molded hollows on the top and bottom to allow for pointed spikes to be installed that assist in fixing the spacer body in place between is vertebra.

3. An interbody spacer body as in claim 1 that has a construction in which the rotateable blade pair are on an axis which only connects on one side of the spacer body and where the shaft that is turned by the user.

4. An interbody spacer system as in claim 1, in which the blades are constructed with a chord-wise twist, defined similar to a shape defined similar to a radially jack screw thread pattern.

5. An interbody spacer system as in claim 1, in which the spacer body has a hole extending through the spacer body on the same side on which a blade shaft is affixed, said hole is threaded to allow for instrument mounting on the spacer body.

6. An interbody spacer system as in claim 1, in which the spacer body has an unthreaded hole extending through the spacer body on the same side on which a blade shaft is affixed, said hole large enough to allow medical personnel to insert instruments into the hole when the blades are turned to the vertical position.

7. An interbody spacer system as in claim 6, in which the rotating blade pair closes off holes in the spacer body, allowing medical personnel to fill the hollow of the spacer body with medically helpful fillings during installation, but allows access to the filling and the hollow of the spacer body when the blades are turned to their vertical position.

8. An interbody spacer as in claim 1 in which the blades can be locked in place by a sleeve equipped with extension fingers that interlock with the blades and the spacer body when a washer nut is tightened on the blade shaft.

9. An interbody spacer as in claim 1 in which the blades are constructed with holes in them that allow for bone growth and easier connectivity between the interbody spacer and the body into which it is installed.

10. A method of installing an interbody spacer body, comprising: 1) removing a damaged vertebral disk; 2) placing a spacer body in the same physical position place from which the damaged vertebral disk was located; 3) turning a pair of is moveable blades so they extend above and below the spacer body into the vertebra; and 4) locking the blade pair into vertical position by a washer nut which rotates on the same axis that turns the blade pair by driving the nut toward the spacer body until the nut is very tight.
